(19) United States
(12) Patent Application Publication
(10) Pub. No.: US 2004/0267570 A1
(43) Pub. Date: Dec. 30, 2004

(54) Method for information and management system for health care

(76) Inventor: Robert E. Becker, Carrabassett Valley, ME (US)

Correspondence Address: MCCARTER & ENGLISH LLP, CITYPLACE I, 185 ASYLUM STREET, HARTFORD, CT, 06103, US

(21) Appl. No.: 10/830,662

(22) Filed: Apr. 23, 2004

Related U.S. Application Data
(60) Provisional application No. 60/464,623, filed on Apr. 23, 2003.

(51) Int. Cl. G06F 17/60; A61B 5/00

(52) U.S. Cl. 705/2

(57) ABSTRACT

Using the method of invention health care system managers, government authorities, insurers, health providers, can use devices, tests, methods of assessment, software programming or hardware programming or other procedures for determining the health or health related status of a person to reach sufficient precision and certainty in clinical examination and test measurements that the true health status of an individual can be described, differentiated from the measurement error components in each examination and test result and the course of the measurements interpreted meaningfully as part of a physician or patient or member of the public seeking well-being and health adopting a prescribed or self prescribed plan for monitoring health and achieving desired health goals and as part of health care system managers, government authorities, insurers, health providers providing improved controls over the quality and costs of health care. Computer software or hardware programming for these methods and for methods disclosed in the co-pending provisional patent applications hereby expressly incorporated above and below by reference as part of the present disclosure enables the physician, health professional, patient, or healthy user, health care system managers, government authorities, insurers, health providers to establish reliable precision and certainty for measurements in clinical examinations and tests whether used for medical research, patient care, health related activities, management of health care systems. Applications are to provide precise and certain interpretations of health indicators based in the improved precision of measurement, earlier detection of changes in health status for both health monitoring and disease management, to provide statistically grounded evidence for possible causal relations among health interventions, disease processes and the clinical or health status of the person, to provide real-time feedback to health care system managers, government authorities, insurers, health providers about the actual outcomes from treatment of individuals in the health care system and to allow this information to be available in real time to develop clinical decision rules to guide care and management choices in the system.

### CROSS-REFERENCE TO RELATED PRIORITY APPLICATIONS

This application claims priority to U.S. application Ser. No. 60/464,623, filed Apr. 23, 2004, entitled “Method for Information and Management System for Health Care”, the disclosure of which is hereby expressly incorporated by reference as part of the present disclosure.

This patent application is related to the present inventor's following co-pending patent applications which are each hereby expressly incorporated by reference as part of the present disclosure: Ser. No. 10/182/785, filed Aug. 1, 2002, entitled “Method of Conducting a CT that Enables Assessment of an Individual Patient's Response to a Drug or Other Medical Procedure Used to Treat a Condition of the Patient;” Ser. No. 10/182,878, filed Aug. 1, 2002, entitled “Method of Assessing an Individual Patient's Response to a Drug or Other Medical Procedure to Treat a Condition of the Patient Using a CT;” Ser. No. 10/213,305, filed Aug. 5, 2002, entitled “Method for Reliable Measurement in Medical Care and Patient Self Monitoring;” and Ser. No. 60/391,492, filed Jun. 25, 2002, entitled “Method for Reliable Measurement in Medical Care and Patient Self Monitoring.

### FIELD AND OVERVIEW OF THE INVENTION

The present invention is directed to a method of using statistical and scientific knowledge and theory that provide precise and certain assessments of health status as indicated by commonly employed measures of health and illness, medical examinations, medical tests, scales whether self administered or administered to the individual being tested, medical or other human activity monitoring instruments, or any other form of health related assessment to allow real time management of individual patients and citizens who use health care and to provide real time feedback information about care outcomes to health care system managers, government authorities, insurers, health providers to manage the health care system for quality and cost controls. The present invention differs from current practice: current health related and professional medical assessment methods do not establish the test-retest precision and certainty of clinical examination and test measurement for the subject, do not develop a plan of assessment out of studies of reliability and certainty of clinical tests and examinations, do not provide real time feedback to clinicians of information about the outcomes of treatments and diagnoses in the health care system and do not provide real time feedback to health care system managers, government authorities, insurers, health providers of information about the outcomes of treatments and diagnoses in the system. Current methods do not provide for a plan of assessment with sufficient clinical examination and test test-retest precision and certainty to optimally use the information from assessment as indications of the patient's or user's actual progress towards health goals. In our medical and personal health applications of scientific and medical scales, tests and examinations we lose information because we depend on personal and professional judgments to interpret how correctly the tests, scales, examinations, measure the true condition of the subject. We lose control over the quality and costs in the health care system because we cannot access data from individual patient treatments or diagnoses since the data are in multiple charts not aggregated, of unknown precision and certainty, open to systematic errors since the conditions of diagnosis and treatment do not reference the same guidelines and procedures and adherence to guidelines or current standards of scientific medical knowledge is not indicated or evaluated. The present invention specifically addresses each of these deficiencies in current methods of self and medical, health or treatment information monitoring and management of a health care system.

The method of invention in applications to self-care and medical care information and management systems differs from current practices. Currently we disseminate health information, guidelines, and new medical findings in articles, publications, broadcasts, advertisements, and so forth, as information not in practically useful form—not presented as practical interpretations. The information is not useful fully due to the time limits of practitioners and lack of technical knowledge of the public. This information overload does not integrate the new findings and their normative, health engendering, therapeutic or quality and cost system management implications for health care system managers, government authorities, insurers, health providers with the applications to individual patients or by a person in his or her own life. The user, manager, reader or listener must interpret the meaning, implications, and methods of application of research for his or her own health and for the health of others. The method of invention overcomes this need for personal or professional judgments to interpret and manage new standards of health and treatment for use with individuals. Under the method of invention new findings can be integrated by providers, developers or manufacturers of measures of health and illness, medical tests, scales whether self administered or administered to the individual being tested, medical or other human activity monitoring instruments, or any other form of health related assessment such that the medical information becomes a grounds for interpretation of the reliable assessments of health status and results can be aggregated among users. It is anticipated that this information transfer will be ongoing; a new clinical decision rule guideline and a new article or other announcement of a medical advance will be provided to the user in a form that integrates the new information into the device or system for interpretation so the patient will have a more direct, personally relevant, specific interpretation of the latest medical and health information in terms of the import for the patient his or her self and the system managers will have real time information to support quality and cost related decisions.

The method of the invention uses the model that tests the precision and certainty of any medical or health assessment, takes into account the health or medical goals of use, develops a plan of assessment adequate to provide the reliability required for the assessments to be useful indicators of progress towards health goals, provides a display or output that interprets the current measurements from scales, instruments, methods, systems, in relation to the patient or user aims, provides a program or processing system that interprets the status indicated by the processed assessments, and updates the interpretive database with new medical or health advances for the citizen's private use, for health practitioners, for system managers.

The method of invention establishes the precision and certainty of a health or clinical measurement by determining the standard error of measurement for the data set developed by the user of a test, scale or examination.

The method of invention develops confidence intervals of measurement for health and disease uses of clinical examinations, tests, scales, or other measurements and compares the adequacy of the precision and certainty of single and combinations of multiple administrations of the examination, test, scale or other measurements to select a measure with adequate reliability to achieve the clinical or health monitoring purposes of the user.

The method of measurement uses the reliabilities of measurement expressed in confidence intervals of measurement or otherwise as needed for the application to distinguish statistically significant deviations from a projected health or clinical course measured by the indicator, to provide reliabilities for the deviation from the earlier course, and to support the clinical or health interpretation of the changes in measurement.

The method of invention uses confidence intervals of measurement and the above mentioned methods of reliably detecting deviations from a predicted course as test criteria for hypothesis testing in an n-of-1 trial. Consequently, n-of-1 trials become more practically available to health professionals and the public to evaluate health and disease interventions.

The method of invention uses a calculated measure of informativeness defined as the reduction of uncertainty associated with the information becoming available to compare the adequacy of the different methods of processing measurements, of designing research, experimental, or observational studies, clinical trial designs, for the clinical purposes or health purposes or aims of the health professionals in patient care or well-being or health activities of individuals.

The method of invention uses software programming or hardware design for a computer, data processing device, or other device to make these methods available to users.

Using these resources the individual pursuing health goals or the professional health care provider can quickly detect effects on health status from interventions or changes in health habits or practices and can gather evidence of the importance of the intervention or changed practice to health status changes.

Using these resources the health care system managers, government authorities, insurers, health providers pursuing quality and cost controls in health care can quickly detect effects on health status of the individuals in the population using the system for the diseases, health conditions, disabilities, treatments addressed in the system.

As may be recognized by those skilled in the pertinent art on the teachings herein, the method of the present invention is applicable to the development, registration and use of any measures of health and illness, medical tests, scales, examinations, whether self administered or administered to the individual being tested, medical or other human activity monitoring instruments, or any other form of health related assessment where without the benefits of the method of invention the user must rely on clinical or health judgments to interpret the health or treatment implications of an assessment with the data aggregated in or near real time and analyzed and interpreted to provide precise and certain indicators of quality and costs in the system. The invention provides scientific and statistical research based grounds to self-care health and medical evaluations, assessments, decision making, treatment and management by health care system managers, government authorities, insurers, health providers. The invention, in new applications to individuals and health and disease monitoring and management, uses statistical and scientific arts widely practiced to study groups of patients and in biological research.

### BACKGROUND OF THE INVENTION

Each individual, and each physician, must assume that his or her methods of personal health assessment or professional clinical methods of assessment have sufficient reliability and validity—are sufficiently free from random or systematic error from one administration to another and express the actual or true condition of the person. Any rational system of decision making is only as strong as its weakest link. When judgments about personal health, or a physician's clinical judgments, are grounded in unreliable or inexact measurements the conclusions and decisions lose validity. Self-care for health and physicians' medical care of patients must be grounded in reliable and valid individualized assessment of an individual's health status and clinical response to disease or treatments. The method of invention provides statistical and scientific grounds for personal, physicians' and other health care providers', and for health care service or funding organizations' decision making in areas where now in self-care, medical care, and health and disease management and funding for health services, or other health related services we depend upon the “unsystematic” clinical experiences and judgments of professionals and the personal judgments of individuals providing self-care. (Guyatt et al., 2000) With these newly available resources health care system managers, government authorities, insurers, health providers have real time access to information needed to manage the health care system. Currently health care system managers, government authorities, insurers, health providers depend on clinical trial, observational studies, epidemiological studies, and other research designs separate from clinical practice to gain the information to assess the efficacy of diagnoses, treatments and other procedures in clinical practice. The invention provides real time information to evaluate clinical practice.

We illustrated the problem of how a state of the art medical assessment used by trained experts may not provide sufficiently precise and certain measurements of the patient's true condition to be grounds for health care decisions and management. Yet physicians use these methods of medical assessments for medical care decisions, and persons in self-care use similar methods, without controls for the errors in measurement. Becker and Markwell (2000) show the error in the tests used to assess the cognitive status of Alzheimer's disease (AD) patients is sufficiently large to obscure both the short term decline in cognitive performance typical of the disease and treatment effects. This leaves the patient and the practicing physician no reliable clinical assessments of individual patients to inform clinical judgments of probable future status or the effects from treatment interventions. In many health and medical conditions the methods of assessment have unknown precision and certainty. An assessment, test, scale, or examination used without taking into account the necessary conditions to assure reliability is an unreliable indicators of current health status, changes, or effects from changes in health habits, practices or treatments. Weights, blood pressures, blood glucose assays, exercise measures, physical performance assessments, scales for mood or cognition or other bodily states, like all measurements have both systematic and random errors that make the measure of unknown reliability. For decision making in patient care and system management to reach a given level of certainty the elements that go into the decision making must each have sufficient certainty and precision such that the decision choice can be depended upon as adequately an indicator of the person's actual state for the purposes for which it is intended.

The error variance in the repeated uses with a person of medicine's clinical examination methods and laboratory procedures, or in personal use of home monitoring or personal methods of health assessment, is not studied scientifically and statistically and the effects of error variance taken into account in each assessment. The method of invention enables the individual providing self-care, or the physician, or others, health care system managers, government authorities, insurers, health providers to conduct and interpret assessments such that the error is taken into account and a course of assessments over time becomes a more reliable predictor for the individual or physician to base health care decisions and for the health care system managers, government authorities, insurers, health providers to base system management decisions.

At one end of a spectrum of reliability, we have no personal or clinical methods of assessment of the patient sufficiently free from error to reliably distinguish changes over short periods of time or changes from treatment from random test error. (Becker and Markwell, 2000) On the other end of the spectrum of reliability even medicine's most reliable assessments—for example laboratory examinations—offer an interpretation based on a normal range of test results which allow 5% (or thereabouts) of all routine observations to be classified as outside the normal range. When the use of health and medical assessments does not integrate a model that takes account of this variable error range among outcome measures both the individual in self care and the practicing physician, the health care system managers, government authorities, insurers, health providers, must resort to personal judgments and guesses about the precision and certainty of the information on which decisions will be based or defer decisions until reliable information is gained in long term research studies.

The n-of-1 trial provides an illustration of the limitations imposed by assessments of unknown reliability. The n-of-1 trial is a method of randomly and blindly assigning treatment and placebo in one individual to ascertain whether the intervention provides a benefit. It is a scientific and statistical design to provide a gold standard for the question any individual interested in personal health asks—“Does this health practice benefit me?” (Guyatt et al., 2000; Larson et al., 1993; Backman and Harris, 1999) Assessments of an individual obtained under blind conditions of sequential treatment by active treatment and placebo are compared to determine the efficacy or safety of the treatment in the individual patient. However, the n-of-1 trial has limitations: the randomization procedure is time consuming; the trial exposes the patient to periods of no treatment in placebo treatment; the trial often has less statistical power than a CT increasing the likelihood of erroneously continuing or discontinuing a treatment on the basis of the n-of-1 trial results or the results being inconclusive. Therefore the clinician will not want to use the n-of-1 trial technique when its use can be avoided. (Johannessen and Fosstveldt, 1991) One source of limitation is that the current n-of-1 trial methods do not call for the reliability of measures for the individual to be established. The n-of-1 trial now uses methods to control error of measurement effects that are used in group comparisons in randomized controlled trials. In randomized controlled trials error of measurement is taken into account by comparing the means of measurements in different patients with the assumption that the random errors of measurement have a zero or equivalent difference in their contributions to the means used for comparisons. As we have shown elsewhere (see the Preliminary Patent Applications incorporated below) establishing the error of measurement and developing a plan of assessment based on the limitation of measurement due to error and the uses of the measurements make n-of-1 trials more practical. Nonetheless for the patient, the clinician, the manager, the n-of-1 trial is needed to determine whether a treatment has more effectiveness than placebo or one treatment more effectiveness than another for an individual patient. The trial results can be guessed or the trial can be scientific. The current invention allows scientific n-of-1 trials to inform clinicians' decisions and managers' decisions.

Using the method of invention the n-of-1 trial becomes a model available to any individual to evaluate the efficacy, or safety, of a health practice or intervention for the individual personally. Without the method of invention and its uses of confidence intervals of measurement, criteria of clinical significance, criteria of statistical significance, (see reference above and descriptions below in methods) decisions must be based on less reliable assessments and interpretations of assessments. Measurements with established reliability cannot replace personal judgments by the individual engaged in self care or clinical judgment by a physician. Measurements of known reliability can better ground all forms of judgment and more directly interact with health care and medical research to provide more exact or accurate interpretations and predications for an individual. The inference that a health care practice or medical treatment applies to a person or benefits a person today depends on the physician's unsystematic clinical experiences and unsystematic clinical judgment which has unclear or no scientific evidentiary support. (Guyett et al., 2000) The individual engaged in self-care can at best be expected to reach the unsystematic reliability available to physicians. The method of invention replaces unsystematic experience with statistically and scientifically derived evidence of reliability.

The method of invention has not been addressed in the medical or scientific literature or in earlier inventions:

In U.S. Pat. No. 5,262,943, Thibado et al., show a system that “manages patient information and assessment.” It generates “reports” and displays “repeated measures data to assist a user in managing and objectively analyzing a patient's treatment.” Thibado et al. do not claim nor address the problem of the reliability of repeated measures and the problems of error of measurement and systematic error interfering with the interpretation of measures and estimating the individual's true, rather than true+error, state. The method of invention claimed herein tests statistically or otherwise the reliability and validity of specific repeated measures as predictors or descriptors of a patient's outcome interpreted using results from medical or health research studies and then uses the reliable measures to inform individual self or patient care. Repeated measures are used, only as required in an assessment plan based on reliability studies with the measures, to provide reliable and valid longitudinal measures of health care status, for prediction of future health care status in relation to the medical and health literature, and for interpretation or prediction of patient responses in treatment in relation to health care research. Thibado offers a “system for managing client information” and the method of invention herein claimed is a system for individuals or physicians to improve the reliability of personal or professional judgments by basing them in statistically and scientifically evidenced reliability of measures.

In U.S. Pat. No. 5,737,396, Garcia, provides pharmacy data to telephone callers. In the method of invention herein reliability of methods of health care assessment are determined and the data can then usefully be made available to consumers and may use telephone or other electronic or nonelectronic means of communication.

In U.S. Pat. No. 5,819,229, Boppe, offers a method to integrate surgical equipment for more effective medical care. There is no evaluation of the reliability of the uses of these devices in contrast to the claims herein.

In U.S. Pat. No. 5,860,917, Comanor et al., describe a method “for evaluating the response of a patient affected with a disease to a therapeutic regimen.” The invention herein researches the reliability of methods of personal health care and physician medical care assessments and the methods of improving reliability of assessment to meet the clinical or personal health care aims. Comanor et al., address the “utility of treatment regimen for treating a patient affected with a disease” by a “model (for) the complex interactions among patient variables” while the invention herein establishes the reliability and validity of a given method of assessment or variable of interest or a set of variables of interest established prior to any personal health activities or research. The invention herein addresses reliability of assessments in individual patients while Comanor et al., rely on the aggregation of group data, specifically active treatment versus placebo, and the statistical analysis of this aggregated group data by “discriminant and logistical analysis” to “model the complex interactions among patient variables.” The method herein, by analyzing the reliability of methods of assessment and the planning of assessment to estimate the true course of an individual over time could not assess the complex interactions among patient variables because data are not aggregated across variable in a manner to allow such analysis with the exception that groups of patients may be use in intermediate steps to establish reliability of assessments in groups to inform judgments about reliability of assessments in individuals. Variables may be combined herein but only within single subjects to provide a reliable single assessment or outcome measure in place of a number of simultaneous independent assessments of the same person. Consequently Comanor et al., focus on characterizing patient care using diseases as a discriminating category. The invention herein characterizes the error of measurement in methods of personal health care assessment or patient care assessments for each individual. As anyone versed in the state of the art recognizes Comanor et al., depend on “a set of predicted outcome values” derived from “discriminate function,” “factor analysis” and equivalent analysis of data aggregated across patients while the invention herein does not use this aggregation of data in research and practice but characterizes the responses of individual patients. Comanor et al., apply least squares statistical methods to data aggregated across patients. In the method of invention herein various forms of least squares statistical methods can be applied to develop the slope of response of each individual in an assessment plan not to aggregated patients' data. Comanor et al., aim to aid the physician “selecting among therapies.” The invention herein aims to aid the physician to evaluate the status of the patient, the individual to evaluate personal health status, reliably and may be used in the efficacy, safety, economics, uses of a specific health practice, procedure, device or drug for clinical or investigative purposes but not as a decision making process for selecting a therapy as inherently more efficacious. Comanor et al., address the problem of assessing therapies the method of this invention addresses the problems of assessing whether a therapy has produced a true change in an individual patient and the magnitude and importance of that change, or potential change, for the individual patient. Comanor et al., address “factors that are predictive of patient's responsiveness to a treatment regimen;” the invention herein does not predict a patient's response to treatment, and therefore its appropriateness for the patient, the invention herein addresses how to reliably determine the response to a treatment.

In U.S. Pat. No. 6,016,345, Quattrocchi, describes a method for patient's to access medical information. The invention herein develops reliable assessments of health and medical status and uses new medical information to reach decisions. Patient access is only one aspect of a possible application of this new medical information.

In U.S. Pat. No. 6,126,596, Freedman, a system to monitor a patient's treatment in conformity with guidelines. The method of invention herein develops statistical and scientific research based evidence of the true condition of a person and aims to replace guidelines and expert opinion and unsystematic clinical judgments or personal decisions about health the developed statistically and scientifically based evidence of the patient's state and the implications for the future implied by medical and health care research findings.

In U.S. Pat. No. 6,168,563, Brown, monitors a patient and based on patient responses provides health information. The method of invention herein is a method of establishing the reliability of assessments of patient's and personal responses or current status to determine the probable future course and outcome and to apply medical knowledge to decision making.

In U.S. Pat. No. 6,188,988, Barry et al., claim a system and methods “for guiding selection of a therapeutic treatment regimen” by “providing salient information to a computing device” containing a “knowledge base . . . different therapeutic regimens (and) . . . a plurality of expert rules. The method of invention herein is a research based clinical or personal method to assess health status and to apply health and medical knowledge in self and patient care decisions. It replaces expert opinion to interpret personal health status with research based models of reliable assessment of self or individual patients and planning assessments to describe the true courses of health, disease, treatment.

In U.S. Pat. No. 6,196,970, Brown, uses data from clinical trials carried out for Food and Drug approval and aims to eliminate “fuzzy” assessments in aggregated data identifying responders and nonresponders. Their method and system addresses “fuzzy” responses by “narrowly structured questions and suggested answers.” The method of invention herein is not limited to questions nor to narrowly constructed suggested answers. The method of invention herein addresses variability in measures or assessments by tests of reliability and confidence intervals of measurements and in assessments plans uses repeated measures to establish the reliability and validity necessary to long-term monitoring health in relation to the health aims for the person.

In U.S. Pat. No. 6,221,011, Bardy, establishes a baseline of patient information but does not use methods to establish the reliability of the information. The herein claimed invention does this evaluation of reliability and then applies the findings to self and patient care.

### SUMMARY OF THE INVENTION

The method of the present invention recognizes, and corrects, the current inability of the individual or clinician, health care system managers, government authorities, insurers, health providers, to manage the health care of the individual in self or clinical practice individually or in aggregate, systematically and rationally because of the undetermined precision and certainty in methods of clinical and self assessment. The methods of invention provides a device(s) or system(s) or combined device(s) and system(s) that the health care system managers, government authorities, insurers, health providers may use as a tool in information and management quality and cost related health care decision making.

Preferred methods of statistical and scientific analysis of reliability of personal health and clinical assessment and background to the science and statistics are provided in the present inventor's co-pending patent applications: Ser. No. 10/182/785, filed Aug. 1, 2002, entitled “Method of Conducting a CT that Enables Assessment of an Individual Patient's Response to a Drug or Other Medical Procedure Used to Treat a Condition of the Patient;” Ser. No. 10/182,878, filed Aug. 1, 2002, entitled “Method of Assessing an Individual Patient's Response to a Drug or Other Medical Procedure to Treat a Condition of the Patient Using a CT;” Ser. No. 10/213,305, filed Aug. 5, 2002, entitled “Method for Reliable Measurement in Medical Care and Patient Self Monitoring;” and Ser. No. 60/391,492, filed Jun. 25, 2002, entitled “Method for Reliable Measurement in Medical Care and Patient Self Monitoring, which are hereby expressly incorporated by reference as part of the present disclosure. Other and supplemental methods of analysis could be used as part of this method of reliable monitoring of personal or patient health status and the effects of personal or prescribed health practices, procedures, interventions, treatments. In broad terms, the present invention is directed to a methods of establishing precise and certain health and disease assessments and using these precise and certain assessments as grounds for applying health and disease management more effectively to provide quality and cost savings in personal health care and in direct health services, better integrating primary prevention and disease treatment and facilitating the use of these improved methods by electronic or other systematic methods for integrating and processing information in order to encourage the applications of research in self and patient care.

### BRIEF DESCRIPTION OF THE DRAWINGS

FIG. 1 is a schematic illustration of the flow of information relevant to delivery of face-to-face practitioner-patient services in a current health care system.

FIG. 2 is a schematic illustration of the effect of a currently preferred embodiment of the present invention on the flow of information relevant to delivery of face-to-face practitioner-patient services in a current health care system shown in FIG. 1.

FIG. 3 is a schematic illustration of the flow of information in a currently preferred embodiment of the present invention.

FIG. 4 is a schematic illustration of the management of indirect and direct health care activities using decisions based on the flow of information in a currently preferred embodiment of the present invention.

FIG. 5 is a schematic illustration of the management of technical support to and methods of provision of health care using decisions based on the flow of information in a currently preferred embodiment of the present invention.

FIG. 6 is a schematic illustration of the effect of a currently preferred embodiment of the present invention on indirect and direct services in a health care system.

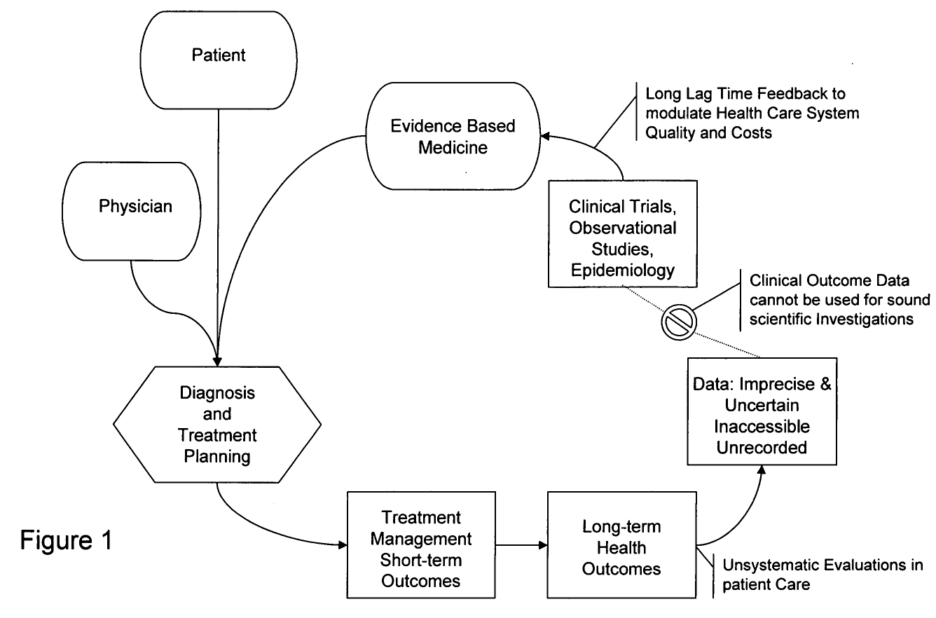

Table 1, Level 0, is a description of the constraints features and benefits associated with the current health care system without application of the methods of a currently preferred embodiment of the present invention to indirect and direct services in a health care system.

Table 1, Level I, is a description of the constraints features and benefits associated with the health care system after application of the methods of a currently preferred embodiment of the present invention to direct, practitioner-patient, services in a health care system.

Table 1, Level II, is a description of the constraints features and benefits associated with the health care system after application of the methods of a currently preferred embodiment of the present invention to direct services (Level I) and addition of interpretations derived from analysis of data obtained in clinical trials and from experience in a health care system after application of the methods described in Level I.

Table 1, Level III, is a description of the constraints features and benefits associated with the health care system with application of the methods of a currently preferred embodiment of the present invention at Level II to indirect and direct services in a health care system.

Table 1, Level IV, is a description of the constraints features and benefits that derive for the health care system with application of the methods of a currently preferred embodiment of the present invention at Level III. These derived benefits follow from the need for improved precision and certainty in health care information demonstrated by the effects of a currently preferred embodiment of the present invention at Level III leading to higher standards for precision and certainty of clinical tests and examinations in clinical trials.

### DETAILED DESCRIPTION OF THE PREFERRED EMBODIMENTS

The present invention is directed to the method of establishing the precision and certainty of any forms of assessment used in health or medical care such that a plan for ongoing assessment takes into account the errors of measurement such that the assessments as used reflect the true condition of the person or patient assessed and this data and long-term outcome data can be aggregated and used to inform the overall management of the health care system. In self care or patient care by a physician the selection of appropriate outcome measures takes into account the reliability of measurement needed to achieve the health aims. This may require that researchers, prior to the public or physician in practice using a method of assessment, use the measure or test at sufficient regular intervals in independent reliability studies or prior to and during health or medical research studies or clinical trials so that a regression line, mean response, or similar scalar summary statistic can be calculated for each individual with adequate reliability to meet the requirements necessary to meet the health care purposes of the assessments. This may also require that the public or physician in practice using a method of assessment, use the measure or test at sufficient regular intervals and calculate the reliabilities of the examinations, tests, scales, or measures, in their use to assure their compliance with the reliability requirements developed in a clinical trial or other scientific medical study or to assure that the examinations, tests, scales or measures have sufficient reliability to be valid indicators of the individual's true health status or sufficient reliability to meet the needs of the analyses or interpretations in which they are used.

As an example persons seeking to reduce their weight to medically recommended levels for their body type can be assessed using the measure weight on a scale once, three times, or more times, on as many successive days as needed to establish the error of measurement in the individual's use of the scale. The individual can then weigh at weekly intervals to establish first monthly periodic cycling or variance and then seasonal or yearly variances. Line fitting to the data, then removing the slope by subtracting the data predicted by the fitted line from the raw data leaves residuals. Means and standard deviations can be calculated for these residuals, the standard deviations are standard errors of measurement. With reference to a table of a Gaussian distribution a confidence interval of measurement to meet any criteria of statistical significance can be calculated.

Using these methods or other methods referenced herein the cumulative evidence of error contributions in a data set is used to adjust subsequent measurements for error by providing a confidence interval at an acceptable level of probability (p=0.95 for the confidence interval is customary in medical research and would thus be appropriate here except when local conditions make a different probability appropriate). As a result of the confidence interval of measurement error single or multiple assessments may be required as part of a plan of assessment. The plan requires numbers and times of weights (or for other variables to which the methods in the example may be applied) based on the reliability needed to display the trend of weight over time with sufficient certainty to allow the user to use the trend as an indicator of compliance with a plan for weight reduction, gain, control, according to the user's aims. A device or system is provided that shows over time the longitudinal course and confidence intervals such that error variance can be used to distinguish random or covariate related systematic or periodic changes from statistically significant deviations from the program course to maintain, to increase, to reduce, or once at the sought weight to maintain, weight. The program or device also displays the course in relation to criteria that express the health aims: in the case of weight ranges appropriate to individuals of this individual's body mass and the probable health outcome changes that accompany deviations from the optimal research evidenced weight. Thus the course of the individual can show a prediction of how soon the current program of change will reach each category of outcome, the relation to the planned course, the long-term implications of deviations from the planned course of weight change. The display will show confidence intervals and thus reassure the user of innocuous random deviations but identify the need for attention when weights, improbable as part of the chosen or actual course, indicate significant deviations. The device or system can also be updated with new medical research and display the implications of the new findings for the individual's current and optional alternative courses over time. Similar devices or systems or programs are developed for other outcome measures, commonly employed measures of health and illness, medical tests, examinations, scales whether self administered or administered to the individual being tested, medical or other human activity monitoring instruments, or any other form of health related assessment. These data are then aggregated in real time and interpreted by health system managers for interventions by guidelines, approvals, restrictions, payment structures or other management tools to assure desired quality and costs in the system. The method is described first and then its implementation in specific assessments and in management.

The method of invention in this application uses the methods of pre-trial reliability studies and methods of deriving an assessment plan described in the present inventor's co-pending provisional applications expressly incorporated by reference as part of the present disclosure above. The method of invention in this application can also use methods of retrospective or ongoing iterative analysis of measurement data accrued to the present to establish the reliability of a set of examinations as described herein. The method of invention in this application can also use the methods applied in pre-trial reliability studies and methods of deriving an assessment plan described in the present inventor's co-pending provisional applications expressly incorporated by reference as part of the present disclosure above but applied to a data set of practitioners' or evaluators' clinical ongoing evaluations of patients or subjects to establish the reliability of a measure, examination, test, or scale in the hands of an examiner or examiners in clinical practice. In the current invention each of these methods are applied to the problems of providing reliable assessments of health and medical status and reaching personal and professional medical decisions in response to health aims or evidence without having to depend on unsystematized personal and professional judgments of unknown reliability. The current invention applies and adapts as the situation requires the reliability methods already applied in clinical trials to personal and professional patient care in situations where clinical trial evidence is not being applied to the individual or where the individualized analysis of clinical trial or other medical or health evidence is not available as a model for personal or professional choice of methods of analysis, study, assessment, or decision making.

The method of invention consists of the following basic steps. A device or system or program processor for data or program to structure design and execution of care using the methods described herein is provided for the user the device or system or program enabling the user to proceed through the following steps:

1. Specifying Health or Medical Aims.

The user specifies the aims for his or her health or disease management program. These aims lead to

a. the choice of a measure, examination, scale, or test that can be defended as validly reflecting the individual's status in relation to the aims,

b. criteria for achievement of the aims by which the individual can determine success or failure reaching health aims and

c. statistical significance criteria or the level of chance occurrence of a measurement tolerable under the conditions of the application by the individual.

2. Choosing a Method of Assessment.

a. All assessment depends on a variable, outcome measure, specific test, scale, examination or other measure that must have validity as a direct measure of a health aim or as a surrogate for the aim. An assessment can be chosen because of its established place in medicine—such as methods of blood pressure measurement, blood glucose measurement, scales of cognitive performance or physical performance-or as an innovation. Reliability studies in groups may be needed to demonstrate adequate reliability and validity for the measure or scale to even be considered as a measure for specific individual health aims. These studies are carried out and then applied in individual care using the methods of pre-trial or ongoing reliability study described in the incorporated applications and herein. Group reliability studies may not be available or the applications of their results may be questioned in which case the provider of health care or disease management will use the methods of invention to establish the reliability of the test or/and the reliability of the test used by the test administrator with a specific subject, the latter two may be the same person. Thus the method of invention calls for the manufacturer or provider of the system or instrument to demonstrate the adequate reliability of the measure for the purposes it is put to in health care as part of the tools provided to the user and or to provide the tools to the user to assist the user to determine the reliability of measures. The user then chooses the assessment and the instrument to provide the assessment and the system, device, or instrument needed to support determining the reliabilities of measures and their interpretation by comparisons to research data or other aggregated patient or subject data.

3. Determining a User Specific Error of Measurement and Covariate Effects.

a. The measurement instrument or system (hereafter called assessment system) provides a program or procedure for the user to take the measurements on repeated occasions such that the test-retest reliability or error of measurement for the user can be calculated. The assessment system can also practice the user with application of the measure until the user's error of measurement falls within the confidence intervals for the group error of measurement obtained following the procedures in 1 a or reaches a level required by the informational aims of study identified under 1. The errors of measurement can be calculated using reliability coefficients, generalizability theory, randomization statistics, as appropriate to the measure and application as would be understood by anyone skilled in the arts of statistics and measurement. The errors of measurement also can be calculated for an individual by removing trends in the data set with line fitting and subtracting the time trend. A mean and standard deviation can then be calculated for the residuals. Since the standard deviation represents the standard error of measurement for the data set the reliability can be expressed and confidence interval of measurement calculated as would be understood by anyone skilled in the arts of statistics and measurement. Calculating a standard deviation for the residuals after removing linear trends in the data set is useful for estimating the confidence interval of measurement for a data set from one individual with repeated testing with a measure and for comparisons across individuals. Combinations of data into descriptive summary statistics and iterative calculations of reliability as the data set is enlarged with new measures are useful for establishing the conditions for an assessment plan that match the aims of the evaluations. Generalizability theory is useful because variance from other variables and error variance can be calculated simultaneously. Randomization statistics are useful where exact probabilities are sought with minimum underlying assumptions about the characteristics of the data set and sources. The methods of determining error of measurement are both known to one skilled in the art and described in the incorporated applications and references therein to earlier work.

4. Setting the Assessment Plan.

a. As described in the incorporated applications an assessment plan is developed and demonstrated by simulation or application to provide reliability for descriptive summary statistics for the individual's course over time. As described in the incorporated applications, according to the intended use of the repeated measures different statistics, means, medians, ranges, slopes, curves, may be appropriate as known to anyone familiar with the arts of statistics and measurement. As described in the incorporated applications, each data point at a given time may require more than one application of the assessment to reach satisfactory reliability that sufficiently narrow confidence intervals of measurement will be found. The assessment plan developed by the assessment system indicates to the user the summary scalar statistic, the frequency of assessment and its summary into data points and the confidence intervals of measurement that will describe the probable limits of range due to error in the course of the individual.

b. Researchers, health care providers, consumers may choose to determine the relative informativeness of different descriptive statistics. Informativeness can be calculated by first calculating the average prior amount of uncertainty over all possible cases in a distribution of possible outcomes for the aim(s) defined for the intended intervention(s). In information theory the average amount of uncertainty is the negative sum over all possible cases of the probability in a distribution times the log of the probability in a distribution for each case. The average posterior amount of uncertainty is over all possible cases in a distribution of possible outcomes for the aim(s) defined for the intended intervention(s) after the information is provided by the measure and its confidence interval of measurement effect is subtracted giving a reduction in uncertainty. The confidence interval of measurement affects the certainty of events and thus by increasing posterior uncertainty reduces the information content available. Thus reducing the range of a confidence interval of measurement will in general increase the informativeness by reducing uncertainty. This measure of informativeness as reduced uncertainty in aims can be used to compare the effectiveness of measures with different confidence intervals of measurement and also to compare the effectiveness of research study designs reaching the aims of the users.

5. Criteria for Achievement of Health Aims or for Clinically Significant Health Effects

These criteria judge the adequacy of change in health status or the desirability of a given health status. They may take different forms according to the aims, measurements, health or disease implications of assessments. Existing scientifically evidenced medical knowledge provides the standards for the criteria. The hierarchy of scientific sources for medical knowledge is discussed in evidence-based medicine. (Guyatt et al., 2000)

a. Normative or Idealized Criteria.

In some areas of self-care or patient management a population based or research based limit provides a criteria for judging the health status. Examples are blood pressure where specific upper limits are set as healthy. With these criteria the patient is categorically ill or well although subcategories of risk and borderline categories can be defined.

b. Change Criteria

In some areas reduced deterioration in the patient's condition or a trend towards an optimal health status may provide criteria. An example may be weight change in obesity where rate of change or a number of pounds lost each month provides an intermediary criteria until the patient reaches an optimal or acceptable range of weight.

c. Range Criteria

In some areas a range of measurements for the individual may describe desirable optimal health status on a variable and adjacent ranges relative changes in outcome risk. An example is weight where for each body mass medical research can define optimal levels associated with positive health outcomes and for weights above or below increasing risk of negative health outcome.

According to the state of health or disease or the assessment an outcome is provided and updated preferably with new medical research findings. The assessment system displays the user course in relation to the chosen criteria and using the confidence intervals of measurement and clinical expertise on mediating aims and means to the aims displays the user's probable status at present and at future time points if current change or stability persists.

6. Criteria of Statistical Significance

a. Since all scientific evidence receives only probabilistic support the assessment system provides methods for the user, or physician, to set an appropriate level of chance occurrence. This choice defines the confidence intervals used in the estimation of the true course of the user on the variables measured.

These methods of invention present the self-care user or physician supervisor of care with improved opportunities to evaluate whether a health practice is justified by the changes found with instituting the practice for the individual. The study of error of measurement and covariate effects (such as cyclical changes over periods of time) lead to confidence intervals of measurement for the clinical course plotted from the point measurements or statistical summaries of multiple measurements at each point. An intervention followed by a course over time that deviates from the earlier course can be characterized by a probability of occurrence of the post-intervention course as an extension of the pre- or non-intervention course. If one course better approximates the Criteria for Achieving Health Aims or Criteria of Clinical Significance then that course has validity as a desirable health engendering outcome. The preferred course can be characterized as an improbable random variation from the non-preferred course but the user may wish evidence that the different practices, the intervention conditions, are required to achieve the health aims. Using open random or systematic alternative conditions or blind and randomly sequenced alternative conditions the user can determine the probabilities using the confidence intervals of measurement. An n-of-1 trial is an example. The use of confidence intervals derived from reliability statistics, generalizability theory, randomization tests, and analysis of the reliability of measurement in series applications of a test or examination to an individual allows clinical courses to be compared on the probability of occurrence randomly. Thus the risk or cost of an intervention can be balanced against the probability of losing the effect in reaching judgments about health practices and this choice can be reassessed at any time because the user on an ongoing basis has estimations of the probability of the current clinical or health status occurring under the opposite intervention or non-intervention condition or can readily determine the opposite condition effects.

Pre-application, retrospective, or ongoing use of the assessment measure is carried out in the individual to estimate the error of measurement and confidence intervals of measurement are calculated. This pre-application or retrospective or ongoing study of test-retest reliability is compared with earlier group studies to judge how expertly the measurement is being used or if there are possible other confounding sources of unexpected error. The confidence intervals of measurement are then considered in relation to the demands on measurement made by the aims of the health intervention or modeling. A method of measurement can only be acceptable if the error does not interfere with the estimation of the true status of the patient that is required to interpret the success of the health intervention. In a general example where a specific direction of change will be evaluated a 5% level of statistical significance requires a 90% confidence interval of measurement. If health or disease management course monitoring requires stability of course to be evaluated 95% confidence intervals would be chosen if a 5% chance occurrence is to be distinguished since either extreme of the range may be violated. A point summary statistic that occurs with 5% chance expected occurrence is identified as statistically significantly different from those within the confidence intervals. The Criteria for Optimal Health or Clinical Significance are indicated in a display or taken into account to monitor outcome.

A clinical course is plotted from repeated measures over time and the confidence intervals of the course indicated. The course indicates progress over time towards a Range of Optimal Health or Health Criteria or that the individual remains in the required range. It can occur that for example, before time 3.5 the individual shows a course where the confidence intervals do not overlap the Range of Optimal Health—the individual has less than 5% chance that her current health status complies with current medically acceptable criteria for health. After an intervention at time 3.5 the individual's experience falls within the confidence intervals of a projected plan of correction. By time 4 the confidence interval of the earlier course no longer overlaps the current estimated course for the individual—the individual is assured that there are 19 chances out of 20 that the intervention is having the desired corrective effect on her original health state. At about time 7 the individual reaches the Range of Optimal Health and then adjusts the intervention to maintain this level of measurement. During the correction and after reaching the Range of Optimal health deviations of single point summary statistics from the overall course fall within the confidence intervals of measurement and the individual is reassured that the overall plan has not been compromised. A measure outside the range of the confidence interval of measurement expected to occur one chance in 20 may be followed by subsequent measures within the confidence intervals of measurement and while in itself improbable it does not evoke a change in the plan of correction.

Specific applications of the method of invention are given as follows with the modifications required by the specific application listed for each. Unless otherwise specified in each area the same basic method is applied for the variables appropriate in the area. The user specifies a health aim based on medical research evidence provided in the device or system or known to her from reports, chooses a method of assessment, determines the specific error of measurement and confirms with the system that the user specific error of measurement falls within the distribution of error of measurement determined in trained users. Based on the aims, a weight change or range to be maintained, the system calculates the number of measurements required at each point and using data from the group test-retest reliability study determines the assessment plan for the user such that the measurements do not interfere with each other or produce interfering carry-over effects. The user adopts from research evidence provided in the system or from external sources criteria for achievement of these health aims or for the clinically significant health effects called for in her aims. The user then adopts from information provided in the device or system or from her physician or personal choice criteria of statistical significance for confidence intervals of measurement and for judging research evidence used to set aims or criteria for aims. The system provides criteria of statistical significance customary in scientific medical practice as known to anyone skilled in the art as default criteria for the system or device. The system then records user measurements over time and for predetermined periods (menstrual cycles entered for each user, seasons, years, and so forth) determines what cycling of measurement occurs as a covariate of time. The system then displays the summary scalar statistic of the course of measurement over time, the confidence interval of measurement for error and for cycling variations, the criteria for achieving health aims, the time points of interventions and any probabilities of courses in relation to each other as described above in examples below.

For each of these disease or health management systems the data from individual patients can be aggregated and interpreted for relations between immediate clinical results from intervention, or intermediate term results with long-term health outcomes. This data can be used to identify problems and issues in the health care system, apply health solutions to addressing these issues, to understanding barriers to quality and cost control and how to overcomes these barriers, to develop recommended courses of action to Disease Managers, Health Insurers, Government Planners. It is particularly aimed that the methods of invention address current problems & issues in health care. These include, but are not limited to:

1. Delivering Quality Care to all Citizens

2. Controlling Runaway Costs of Health Care: Especially Drug Costs which could Bankrupt the Health Care System

Inappropriate public and professional perceptions of new and costly as desirable features recommending diagnostic and treatment technologies: a new prescription drug reaches market every 4 to 5 days in western developed countries;

Controlling Overuse and Inappropriate use of Drugs Due to Over-Promotion by Pharmaceutical Companies

Knowing when drugs are essential to maximum benefits for a patient to assure the best outcomes; when drugs offer no benefit and only potentials for harm and waste of health dollars

3. Less than Fully Effective Mechanisms to Manage Medical Care for Quality and Cost Controls

Fragmented data and information systems: problems sharing data among practitioners, patients, disease managers; lack of continuity of care; lack of information needed to measure and track results from health interventions at every level-self-care, physician, prevention, management;

Retraining and updating health professions to use new technologies wisely, effectively, in the patient's best interests and economically

Under use of trained health professionals: home health providers; clinical pharmacists; case managers; nurses, physician assistants.

4. Insufficient and Unequal Access to Comprehensive Health Care

Developing an environment where citizens can be fully informed about their health care: Informing citizens for effective interventions to reach healthy life styles—focus on wellness; sufficient information for citizens to hold providers accountable for what happens in health care

Dealing with epidemics from modern life styles and wealth: smoking; physical inactivity; obesity and its consequences; HIV; TB and other antibiotic resistant infections; a health care system over-focused on treatment to the neglect of prevention; . . .

Under use of technology by the health care industry leading to medical errors, misinterpretations of clinical examination and test findings, avoidable drug interactions, avoidable patient non-compliance, decisions not based on the latest medical knowledge, physicians demoralized by paperwork demands, prescription errors, impractical and therefore underused Decision Support Tools currently available to practitioners.

The proposed solution provides Health Care Management organizations with the following benefits: improved management of health care quality & costs because managers have access to more precise and certain information about clinical and health outcomes as they occur, that is improved management of patients by physicians, other health practitioners, patients themselves provides management organizations (Governments, Insurers, Providers) timely information for monitoring and managing quality and cost focused interventions. The system also will improved access to comprehensive health care resources because it supports better integration and balance between direct patient care and primary prevention of disease. More effective use of information technology resources because data used are precise and certain rather than as currently dependent on unsystematic and uncontrolled clinicians' and persons' judgments guide and support health care decisions. The information and management system based on the clinical care supports health management modules results in more effective introduction of new medical knowledge and technological resources within the patient care process and within the health management sector of health care. The system provides interpreted medical knowledge, not uninterpreted medical information, to better update practitioners and the public on medical advances and their economical use in health care. The system can provide interpreted medical knowledge without infringing on the practitioner's individual preferences for clinical practice because both managers and practitioners can set the conditions—such as criteria for a clinically important effect, hierarchy of acceptable scientific or clinical evidence, criteria of statistical significance—to govern interpretation of medical knowledge and clinical findings, to select medical knowledge sources, and to compare different criteria and the results for interpretation.

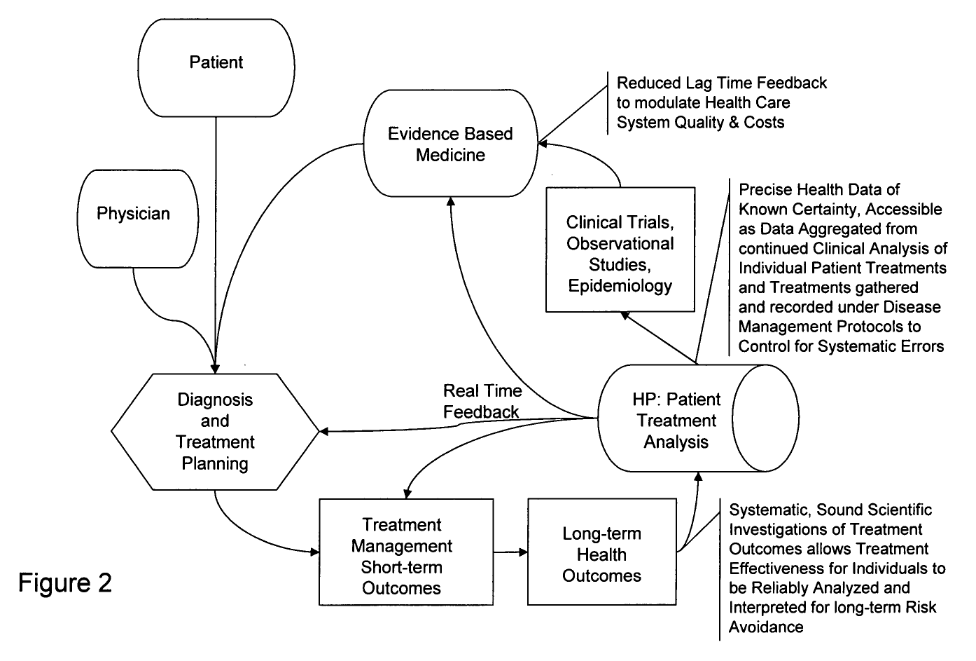

The proposed Information and Management system overcomes many current barriers to quality and cost control. One problem is the clinical practice-medical research information rift. The health care system, as a system, is a cybernetic information system or a dynamic system dependent on feedback about its current state to control the functioning of the system. To provide that feedback in real time, and not years later in results from separately designed and executed research studies, we need to use medical research in real time within clinical practice—research used in real time, more immediately, more efficiently, more effectively, more directly, more often as routine resource in clinical practice—to promote quality health care and cost controls. Currently clinical practice outcome data are regarded as inadequate for hypothesis testing! Why? Because of unknown precision, unknown certainty, inaccessible, data collected in contexts that introduce systematic errors. The methods of invention correct the problems illustrated in FIG. I to produce, within clinical practice, quality patient care and outcome data for real-time feedback controls over clinical practice, to improve the quality of care decisions reached by practitioners everyday and to reduce the lag time to implementation of change based on everyday information about the outcomes of treatments available to managers as illustrated in FIG. 2.

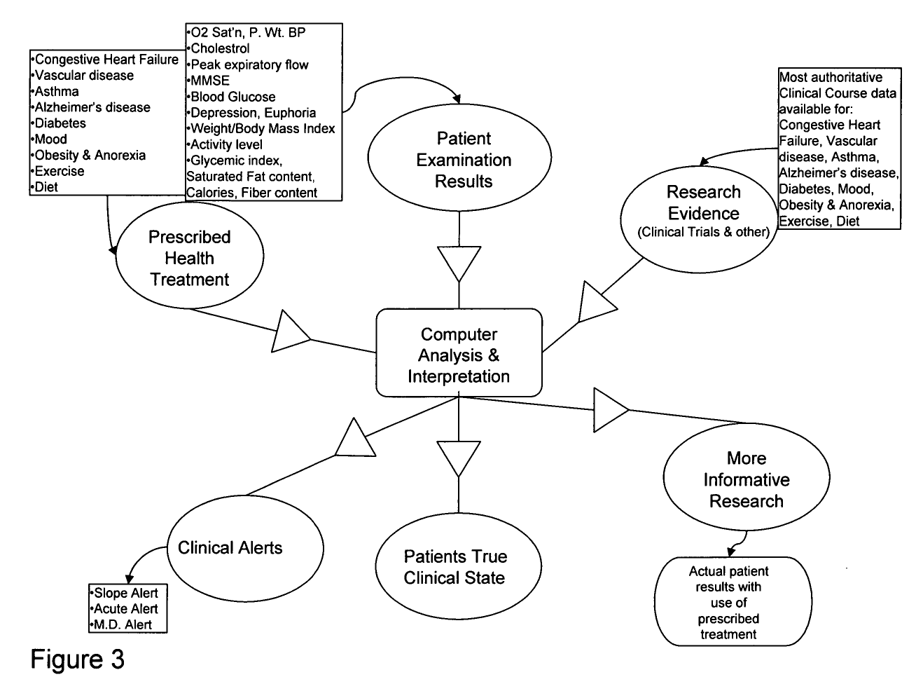

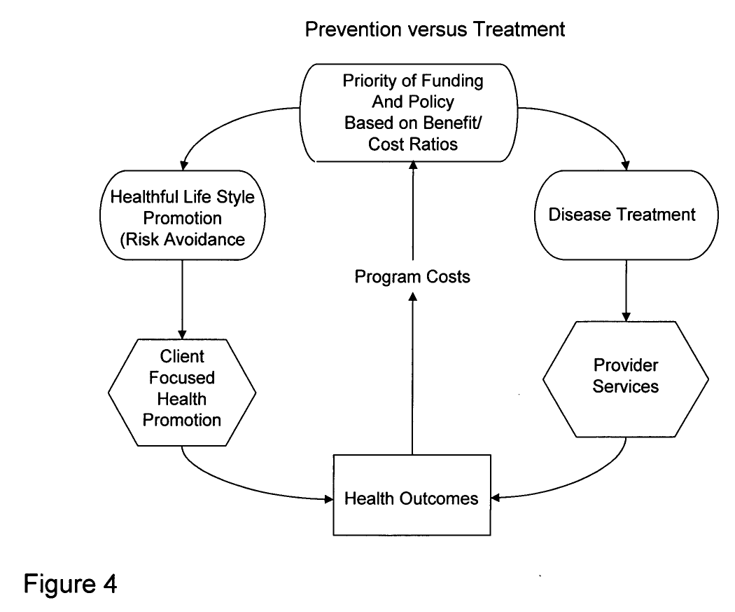

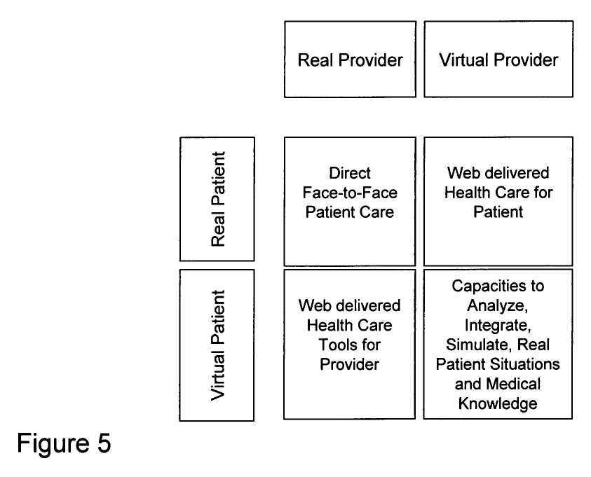

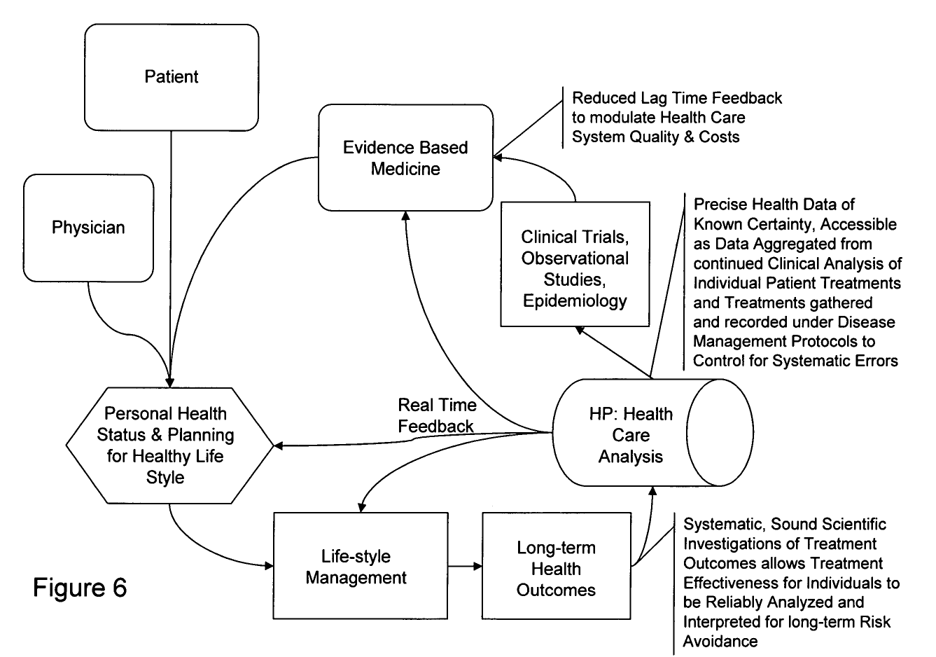

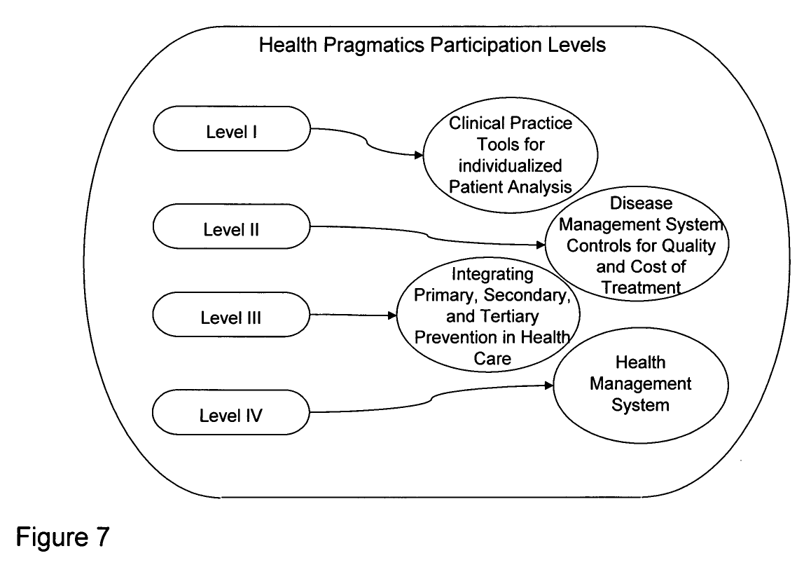

FIG. 3 illustrates this process in the individual decisions in patient care just as FIG. 2 illustrates the improved management process using aggregated information from individual decisions. In this stably controlled, self-modulating, self informing system, clinicians and managers use patient care experiences to generate and test clinical practices. Medical practice is evaluated and managed reliably in real time. Medical research studies confirm clinically tested health management hypotheses and practices and continue to investigate new frontiers in research. As a result the system managers better integrate primary (disease prevention), secondary (disease treatment), tertiary prevention (management of complications to avoid handicaps) in health care. The current health care system overemphasizes disease diagnosis as the ticket for admission to health care which distorts and undermines the effectiveness of health care and cost controls. For example, what will the 21^{th} century epidemic of obesity mean for the world's health care systems? Obesity leads to secondary diabetes which leads to expensive complications. As FIG. 4 illustrates the health care manager with information from each area of prevention can manage expenditures and policies to optimize the overall effectiveness of health investments. As FIG. 5 illustrates the information and management system integrates computer-based or virtual or internet provided health care services and supports with current direct and self care services Today, health care suffers from a hegemony of the provider-patient face-to-face contact. Health care providers and the public are overwhelmed by an information avalanche such that in medicine computers and the internet create their own limitations since human beings under information processing and time constraints can only attend to a fraction of available and relevant information in many situations. The invention plans for usable provider and system supports to be delivered in the virtual world. The invention empowers the citizen with interpretations versus overpowering the consumer with information often of unknown origins or precision or certainty. The data aggregations integrated with the capacity for simulations, information interpretation, management interventions for quality and cost control depend on the precise and certain clinical examination and test data developed by the methods of invention and applied in clinical practice (FIG. 4) and system management (FIG. 2 and FIG. 6).

The methods of invention also address educational requirements of modern, fast-advancing, scientific medicine. Current medical education allows excessive lag-time between introducing medical innovations and changing clinical practices. This produces hysteresis in the education-practice implementation curve. The method of invention enables life-long learning—self-shaping of health behaviors—with real-time, in-office, in-home, problem based learning. In concert with modern educational, practice, management, and research procedures in medicine the methods of invention build towards a problem integration of medicine:problem-based care and records; problem organized, evidence-based clinical resources; real-time problem based learning in the care process; problem analyzed health care management.

These methods of invention technically rest on the problems of errors in science and everyday life—systematic & random error. The invention recognizes that we live in a “fuzzy world;” there is a limit to precise measurement below which we cannot obtain reliable data. In systematic error all of the individual values are in error by the same amount: errors of calibration of instruments; personal errors; experimental conditions; imperfect technique. Researchers and clinicians use similar methods to control systematic error: care to avoid conditions other than the treatment interfering with outcome measurements or invalidating conclusions drawn; comparison of findings with measurements using different methods unlikely affected by the suspected error source; calibration of tests and instruments against a standard if available. In guarding against systematic error clinicians and researchers learn from each other. This mutual learning and support does not exist for random errors.

Random errors occur when a given measurement is repeated and the resulting values, in general, do not reproduce exactly. These include: errors of judgment; fluctuating conditions; small disturbances; definition—for example cognitive performance is not an exact quantity. The experience and discipline gained controlling random errors in research settings cannot be directly applied in patient care. In clinical trials researchers remove random error effects by averaging responses of individuals assigned to the same treatment condition and then compare group averages. Clinicians interpret a clinical examination or test as an indicator for an individual patient's clinical state without knowing the exact magnitude of the true and error components of the score. The random error problem has not received systematic study in medicine except for the study of Becker and Markwell (2000) using Alzheimer's disease patients. Clinicians, to evaluate error effects on their clinical judgments, reference training, experience, expert opinion, or reliability reported in research studies. As a result we can conclude that no currently available source provides scientific grounding to the clinician's distinctions between true and error components of clinical examination and test results. (Becker and Markwell 2000). This research shows that the standard deviation of test-retest results in the presence of relatively high test-retest reliability can produce high error components in clinical examination and test scores. Therefore high test-retest reliability does not guarantee that a clinical examination or test is a true indicator of an individual patient's clinical status. In clinical trials averaging measurements from a group results in the random errors associated with the individual measurements taken for each subject become randomly distributed around the mean calculated from the individual measurements of subjects. As the n increases for the group the random error for the mean approaches zero. Nonetheless for controlling random errors in clinical practice clinicians do not have the luxury of averaging out individual errors in a mean calculated for a group of patients. The methods of invention provide statistical certainty and precision: for precision—establish a Confidence Interval of Measurement (CIm) for the results from a clinical examination or test; for certainty—adopt a Criteria of Statistical Significance to give clinical judgments a known certainty of precision. One then sees that the usual representation of a clinical course through time—the fitted line—misleads. Representing a patient's clinical course by the mathematical representation of a “line” misleads us to regard our information as more precise than it actually is, the line is a misleading indicator. In contrast, applying a CIm by the already indicated methods represents the true course by an area with upper and lower limits. Random errors fall within the 95%, or statistically chosen level of certainty, with an interval with statistical certainty. Under these conditions information may appear “fuzzy” but is closer to the truth. “The ‘95%’ or other probability related Confidence Interval of Measurement is a cautioning indicator.

We can show in an example what measurement error controls with a CIm using the methods of invention imply for medical practice and health care management. Consider the patient D. D. (name changed). The British National Health Service NICE criteria(pages 12-13 available at www.nice.org.uk/pdf/ALZHEIMER_full_guidance.pdf) state: “people with mild and moderate Alzheimer's disease . . . should be examined using the mini mental state examination . . . The assessment should be repeated usually 2 to 4 months after reaching the dose of the drug that is considered suitable to maintain control of the symptoms (the maintenance dose). After the assessment, the drug should only be continued when there has been an increase in, or no decrease in the MMSE score, together with improvements in behavior and/or functioning. This is because not all people taking these drugs benefit from them. For those who do not show improvement, or a slowing down of the disease, in the first few months, it is unlikely that they would show any benefit later on and medication should be stopped.” They also state “Sometimes drug treatment is stopped for a short period in order to determine whether a person is benefiting from the drug treatment” explicitly endorsing the n-of-1 trial but without calling for scientific controls over the trial methods.

In our example patient, between the single last pre-treatment MMSE and the single six-month MMSE DD loses one MMSE point. Under British NHS-NICE criteria medication would be stopped.

Becker and Markwell (2000) found a 90% CIm for single MMSE comparisons of +/−4.16 to +/−4.89. This implies conservatively that due to measurement error Douglas Default's true six-month score could be anywhere between +3.16 and −5.16. In our clinical trials D D had three pre- and three post-treatment MMSE assessments. In the comparisons of means of three MMSEs DD is a probable responder against our research criteria for a clinically important effect—50% reduction in the rate of untreated average decline. These criteria were chosen after consultation with research patients and families as marking clinical effects significant to them. The methods of invention would call for very different treatment of DD with clear implications for other patients and for the policy and funding decisions of managers in Alzheimer's disease health care.

The CIm is calculated using the standard error of measurement (SEM) which reflects current practice, for example reporting educational test performance. Following that precedent the methods of invention use the SEM, or a multiple of the SEM, to estimate the probable limits of the true scores of persons with any given observed score. The methods of invention use the SEM in interpreting score differences. Taking SEM into consideration guards against uncritically accepting small differences between scores as true indicators. If means do not provide adequate precision other practical summary statistics—medians, means of medians, biweighting, removing straggling tails—can be explored. (Mosteller and Tukey 1977).

Calculating the SEM:

1) SEM computed as SDₜ\sqrt{1−rₙ}

2) In which SDₜ is the standard deviation of the test scores and rₜₜ the reliability coefficient, both computed from the same data set. (Anastasi 1988)

3) SEM as the standard deviation of a distribution of scores over measurement occasions. This calculation can be used for a set of multiple retest scores for an individual. Since the sample variance is, on average, smaller than the population variance a correction to the standard deviation must be made to remove bias from the SEM estimation. This correction requires multiplying the squared standard deviation by N/N−1 to obtain the unbiased sample variance. N are the number of observations in the data set. The square root of the unbiased sample variance provides an unbiased estimate of the SEM. (Hayes 1963)

4) Trends can be removed from a data set. Residuals, after regression removes systematic trends, can be studied using the above methods.

Calculating the Standard Error of Difference:

To calculate the CIm the SEM(s) is used to determine the standard error of difference for comparison of two scores. The standard error of the difference (SE_{diff}) between two scores is two times the square of the SEM if the SEM does not differ between the two scores—if SEM₁=SEM₂—or:

SE_{deff}=/\sqrt{(SEM₁)}²+(SEM₂)²

Therefore for analyses the SE_{diff} can be calculated from the SEM based on the data set analyzed unless a second SEM for a comparison condition is available from the data set analysis. Examples where two different SEM might be available are comparisons at two different times when the interval results in sufficient deterioration that the SEM changes because of the patients' deteriorated state or a treatment changes the SEM.

Calculating the Certainty of Judgments: The Criteria of Statistical Significance:

The CIm is developed in response to the level of confidence desired. To provide equal confidence in clinical and research judgments we set p=0.05 as criteria for statistical significance. When the direction of outcome in an application of the CIm can be predicted a 90% CIm meets this criteria. When the direction of outcome change cannot be predicted a 95% CIm must be used. The required CIm is calculated by multiplying the SE_{diff} by the z score for the desired cumulative probability: 1.65 for a 90% CIm; 1.96 for a 95% CIm, or according to the criteria of statistical significance for the comparison.

How do the Methods of Invention Serve Clinical Aims?

In Alzheimer's disease, as in many other diseases, immediate outcome measures serve mainly as surrogate measures to predict long-term health outcomes. For example, in diabetes blood glucose predicts acetylated hemoglobin predicts neuropathy, kidney failure, blindness, peripheral vascular disease, and so forth. In Alzhiemer's disease, as the NICE guidelines recognize, immediate response to drug hopefully predicts intermediate and long-term rate of cognitive and behavioural decline, time to needing full time supervision, time to bedridden, time to nursing home placement, time to death. The methods of invention aim to provide, at a chosen level of certainty, adequate precision to rationally and scientifically manage long-term treatment of Alzheimer's and other diseases. The methods of invention require only precise measurement within the requirements of clinical distinctions demanded of the physician: at a chosen level of certainty—provided by the selected criteria of statistical significance; at an adequate precision—selected on practical grounds. For example using our research and the methods of invention ‘The Alzheimer's Disease Management System works well enough to rationalize patient care.’ This system, a series of management strategies, provides ‘improved clinical tools:’ Management Sequence I—Evidence of effectiveness with initial treatment; Management Sequence II—Detecting deterioration at the limits of measurement; Management Sequence III—Detecting clinical effects from drug when effectiveness is questioned; Management Sequence III Clinical course with overlay—comparing a patient's course to other patient courses, from clinical trials, already treated patients, under different drug or dosing, and so forth.

The methods of invention in addition offer an Information and Management System that is integrated into the clinical care resources on four distinct levels (I-4) of science-based support to clinical practice and health management. A health care system can participate at any or all levels; with precise and certain data from clinical examinations and tests Health Pragmatics helps clinicians and health managers overcome the only restraint upon implementation of these new tools as shown in an example of an implementation of the methods of invention in FIG. 7. In FIG. 7 level I proposes to implement web-based analysis tools for clinical practitioners. These tools assist physicians in evaluating treatment results for one disease such as Alzheimer's. This-allows time for health authorities to respond to issues and problems from the implementation with little chance for negative impact. It is an introduction into a disease area where problems of quality and cost concern practitioners. This approach eases the public acceptance and prepares users for the addition of other disease and treatment programs. After successful implementation, the system can then be expanded to provide the same tools for other diseases or other treatments using experience gained in the initial application. In Level II, tools are added that allow insurers, providers, government authorities to monitor and evaluate in real-time data collected by all treatment programs supported by the methods of invention. This information and management resource provides health authorities with an data needed to manage medical care for quality and cost control. The system provides improved hypothesis generation about treatment efficacy for research investigations and allows for more effective implementation and economical use of preventive medicine. In Level III, functionality is added to begin development of a comprehensive health system that focuses on prevention as well as disease management. The system provides support for self-care with citizen access and monitoring for effectiveness. The system provides evidence-based summaries of medical knowledge, analysis and interpretation of solutions, and simulation for comparing possible decisions for probable outcomes. In level IV a fully comprehensive health system also provides support for improvements in determining the effectiveness of new drugs in individual patients during design and analysis of clinical trials. This provides health policy makers with statistically sound scientific evidence to support guidelines for judging effectiveness of new drugs in individual patients preliminary to approving payment for high cost new drugs proposed by pharmaceutical manufacturers. Insurers and governments who implement HP quality and cost controls as conditions of reimbursement for drugs push pharmaceutical firms to provide more clinically informative clinical trials. By requiring pharmaceutical development companies to determine during clinical trials criteria that identify patients who receive unique benefits with a proposed new drug and those treated equally well with less costly alternatives or those not benefiting from the new drug, care costs can be reduced and quality of patient care improved. The implementation is described in Table I containing Levels O to IV of the methods of invention applied in health care.

The methods of invention can be applied in various diseases, health conditions and treatments. For example:

1. Weight and Disorders or Diseases of Weight

Weight is measured and specific weights targeted in aims and criteria. Based on information from preventive control programs and treatment of diseases and complications associated with lack of control and disease the system managers formulate policies and distribute funding to balance primary and secondary prevention.

2. Blood Pressure and Disorders or Diseases of Blood Pressure

Blood pressure, systolic and diastolic, are measured and targeted. Individual points, summaries of points or closely spaced sampling over periods of time may be used with a monitor. Thus a curve of blood pressure can be plotted and the area under the curve calculated as an expression of total body exposure to blood pressure. The area under the curve of a normal population can be subtracted to produce a scalar summary statistic of excess exposure to blood pressure. Based on information from preventive control programs and treatment of diseases and complications associated with lack of control and disease the system managers formulate policies and distribute funding to balance primary and secondary prevention.

3. Blood Glucose

Blood glucose is measured. A monitor can plot the curve of blood glucose in relation to meals and subtract the area under a normal curve determined in a research study from the user's curve to quantify the excess glucose exposure and, by averaging repeated cycles, specify the times of excess exposure. Based on information from preventive control programs and treatment of diseases and complications associated with lack of control and disease the system managers formulate policies and distribute funding to balance primary and secondary prevention and the levels of blood glucose control sought in the population. Different interventions and prevention can be compared for quality and costs.

4. Cognitive Performance

Tests of cognitive performance are used. The user establishes her own baseline by repeated measures and possible cognitive decline can be estimated by comparison of later measurements to the performance at a younger age. Drug prevention effectiveness can be monitored to select patients who benefit and to not expose patients without benefits to useless treatment, expense, and the risks of drug exposure. Caretakers can find patient assessment tools on web sources to help monitor patients for clinical state and to guide self-care activities.

The method of invention also allows health managers to gather information about the safety of treatments and to monitor for unsafe treatments or treatments that are selectively unsafe for patients under specific conditions of use. At each physician contact or in self assessments the patient, physician, or other enters information about possible adverse events suspected to affect the patient. These data are aggregated and analyzed for deviations improbable based on chance and the improbable deviations flagged and brought to the attention of system managers or providers.

The method of invention can be embodied in a computer software program or hardware system or any device capable of carrying out the required operations or accessible electronically by the Internet or from another centralized source any of these independent of any specific system or method of assessment, or capable of being interactive with devices or systems of assessment, or integrated in a device or method for assessment, or provided as a published set of directions, flow charts, worksheets, instructions, guidelines, technical training, skill training, or other forms and result in publications in articles and books, audiotapes, CD recordings, or other forms of presentation of the methods of invention.

Software or hardware programming includes as needed procedures to accomplish the following steps:

identifying the aims of a clinical trial (CT) or patient care or health care or information and management system. Each of the following references to patient includes a person in self care who pursues well being or health with systematic interventions in health habits or life style. The aims anticipate applications of the CT, or analysis of patient care, in patient care;

identifying proposed outcome measures of each patient's medical condition, and determining whether the proposed outcome measures have sufficient reliability to meet the aims of the CT or patient care and the anticipated applications of the CT or analyses of patient care in patient care;

conducting a reliability study of at least one outcome measure to be used in the CT or to be used or already used in patient care and determining the error of measurement of the at least one outcome measure based thereon;

developing an assessment plan for the CT and or patient care by selecting the frequency and form of measurement of each patient's medical condition based on an error of measurement offering sufficient reliability to meet the aims of the CT or patient care;

identifying criteria of clinical significance for use in the CT and in applications of the CT in patient care;

selecting criteria of statistical significance to set the level of chance occurrence for use in interpreting comparisons in the CT or patient care;

assessing a plurality of patients in the CT or one or a plurality of patients in patient care in accordance with the assessment plan; and further comprising at least one of the following steps:

(i) comparing each patient's clinical course to the criteria of clinical significance, and determining whether the patient's condition is improving or not based thereon;

(ii) estimating the probability that the drug or other medical procedure or health intervention is necessary for improvement of an individual patient's condition by comparing the chance occurrence of each individual patient's clinical course among active and placebo treated patients in a CT or with the use of an n-of-1 trial;

(iii) determining based on at least one long-term outcome of a CT or other observational or other research studies whether the measured improvement will result in a long-term favorable outcome for the individual patient; and

(iv) identifying at least one optimal expected long term outcome, comparing a patient's expected long term outcome to the optimal expected long term outcome, and assessing the probability of whether the patient will achieve the optimal expected long term outcome.

In these steps a reliability study uses test-retest reliability on individual patient data and on data from groups of patients as appropriate to the aims of conducting the test. Determining the error of measurement includes determining the error of measurement of a single administration of an outcome measure and the error of measurement for multiple administrations of an outcome measure summarized as a descriptive summary statistic and the ability to compare the informativeness of different statistics in terms of the aims of patient care.

Each patient's clinical course is characterized by the outcome measures carried out in compliance with the assessment plan. Comparing each patient's clinical course to the criteria of clinical significance includes determining whether each patient meets the criteria of clinical significance and identifying each patient as a responder or not based thereon. The steps of assessing an individual patient's response to a drug or other medical procedure used to treat a condition of the patient are: evaluating the patient in accordance with the assessment plan of the CT or patient care; and further comprising at least one of; confirming that the error of measurement for the at least one outcome measure applied to the individual patient does not exceed the error of measurement for the corresponding outcome measure used in the CT or determined from earlier data from the patient or a group of patients; comparing the patient's clinical course to the criteria of clinical significance from the CT or patient care, and determining whether the patient's condition is improving or not based thereon; applying the criteria of statistical significance from the CT or patient care to estimate the probability that a patient is or will become with continued treatment a responder or not based on the criteria of clinical significance; applying the criteria of statistical significance from the CT or patient care to estimate the probability that the drug or other medical procedure is necessary for improvement of the individual patient's condition; determining based on at least one long-term outcome of the CT whether the measured improvement will result in a long-term favorable outcome for the individual patient; and) identifying at least one optimal expected long term outcome, comparing a patient's expected long term outcome to the optimal expected long term outcome, and assessing the probability of whether the patient will achieve the optimal expected long term outcome.

The assessment plan from the CT or a reliability study of patient care data includes information concerning at least one of: (i) whether different outcome measures reliably support the aims of the CT or patient care; (ii) how outcome measures are combined into descriptive summarizing statistics to meet the aims of the CT or patient care; (iii) how frequently outcome measures or combinations of outcome measure administrations needed to form descriptive summarizing statistics are administered to patients; (iv) how multiple administrations avoid carryover effects; (v) which single measure or descriptive summarizing statistic for multiple administrations is used in data analysis to control error of measurement in a test of hypotheses in the CT or in patient care; and (vi) which single measure or descriptive summarizing statistic for multiple administrations is used in describing the individual clinical course of each patient in the clinical trial or in patient care.

A single measure or a scalar summary statistic summarizes multiple measures taken in relation to each other within a predetermined period of time to form a descriptive summarizing statistic. The selected measure or scalar summary statistic describes the patient's clinical course as a clinically significant response or non-response to the treatment received. A confidence interval of measurement is calculated from the error of measurement and criteria of statistical significance and used to judge the patient's clinical course in relation to criteria of clinical significance. The probability that the drug or other medical procedure is necessary for improvement of the patient's condition includes at least one of the following comparisons: (i) the probability that the treated patient's course would occur under both active treatment and comparison or placebo conditions; (ii) whether a confidence interval of the treated patient's course overlaps or does not overlap a mean of courses within an actively treated or placebo treated group in a CT, (iii) an odds ratio of the cumulative frequency of the treated patient's course among actively treated patients divided by the cumulative frequency among comparison or placebo treated patients in a CT; (iv) an exact probability comparing the treated patient to active and placebo treatment determined by a randomization test; and (v) another comparison required by at least one aim of the CT, patient care, or intended use of the treatment in patient care. Estimating the probability that the drug or other medical o health procedure is necessary for improvement of the patient's or person's condition includes calculating at least one odds ratio for each of a plurality of clinical courses occurring under treatment and placebo conditions in CT data comparisons or for n-of-1 trials with an individual patient. The odds ratio includes the probability that a surrogate outcome indicates a treatment effect will result in a long-term health benefit.

Criteria of statistical significance perform at least one of (i) determining whether an individual patient is a responder or not; (ii) establishing the probability that an individual patient's clinical course could occur under placebo or under active treatment conditions; (iii) statistically supporting the internal validity of the CT, n-of-1 trial or patient care; (iv) selecting confidence intervals; (v) distinguishing as different two or more clinical courses; and (vi) estimating whether a clinical course projected into the future will indicate the patient is a responder or not, is benefiting from active treatment or not, or will have favorable long-term health outcomes or not.

Determining whether an individual patient's condition is improving or not includes at least one of: (i) using n-of-1 trials to confirm whether the patient is meeting criteria of clinical or statistical significance, (ii) using n-of-1 trials to confirm whether the patient is experiencing a clinically significant or statistically significant effect of treatment compared with placebo, and (iii) using n-of-i trials to confirm whether under an alternative treatment condition the clinical course falls outside the confidence intervals of measurement for a course projected from an earlier or later comparison treatment condition. Confidence intervals for measurement of outcomes from treatment, test for treatment and placebo effects in n-of-1 trials.

Determining whether the measured improvement will result in a long-term favorable outcome for the patient includes generating probabilities for long-term outcomes specific to distinct clinical responses. The distinct clinical responses include individual courses, course intervals bounded by confidence intervals of measurement, and comparisons of an individual to others with courses that fall within the confidence interval of measurement of the individual's course. Differences among courses are measured by surrogate outcome variables with confidence intervals of measurement derived from the error of measurement. Confidence intervals for measurement of outcomes can also be derived from treatment or monitoring experience with a person or patient, and a model for a practicing physician to use to assess each patient's clinical course in relation to established clinical and statistical criteria of significance and individual patient courses in the CT.

The programming system also allows a step of conducting a reliability study includes conducting reliability studies of combinations of outcome measures to determine which number and frequency of administrations of the outcome measures is required to achieve the aims of the CT or patient care. Conducting a reliability study includes conducting reliability studies of alternative outcome measures and combinations of number and frequency of administrations to select the outcome measure or measures for the CT or patient or health self care. Comparing each patient's clinical course to the criteria of clinical significance further includes assessing degrees of response in relation to the criteria of clinical significance and the probability of a patient becoming a responder or not if the patient maintains the present clinical course into the future. Evaluating the patient in accordance with the assessment plan of the CT or patient care includes interpreting the results of the evaluation in accordance with the assessment plan and patient data generated in the CT or in the course of patient care or health care. It may be preferable to confirm that the error of measurement for the at least one outcome measure applied to the individual patient does not exceed the error of measurement for the corresponding outcome measure used in the CT or historically in patient care or health monitoring. The system supports confirming whether the error of measurement for the at least one outcome measure applied to the individual patient exceeds the error of measurement for the corresponding outcome measure used in the CT, patient care, health care, or otherwise and if so, determines a confidence interval of measurement for that patient.

These steps are implemented by a system with the following major components or routines and subroutines. An educational or informative module to provide instruction in the system and describe the scientific, medical, statistical, and practical grounding for the system; a demonstration module that illustrated the system's features for the user; a user module that allows the user to access and use the resources of the system. The user registers and establishes an electronic record of data he or others submit and or accesses his or her electronic medical record for the data to be analyzed or monitored by the system. The system provides services to diverse populations: physicians and other health care professionals; patients; families; caretakers; researchers; medical insurers; government agencies; disease managers; pharmaceutical manufacturers; the healthy individual. The resources of the system are specially modified to address the different needs of each of these populations of users.

The system provides two major monitoring and analytic resources: disease or health monitoring; and disease or health management. Monitoring graphs health or clinical indicators over time and uses the subroutines of the system to characterize and analyze the courses plotted. Management similarly plots individual data over time but characterizes and analyzes the data using research data from scientific and medical studies. In both of these activities criteria of clinical significance or health significance, criteria of statistical significance, confidence intervals of measurement can be displayed and data analyzed in relation to these. The confidence intervals of measurement are analyzed from pre-clinical trial, clinical trial, or patient care data and use in conjunction with different descriptive summary statistics and informativeness analysis subroutines to display options to the user. Subroutines also calculate odds ratios, probabilities, distribution frequencies as needed to characterize a clinical course or outcome implications.

For each of the elements or functions in the system for each disease or health management system data from patient care is aggregated with personal identifying information removed and the data analyzed for relationships among short and long-term outcomes, conditions of treatment and problems of safety, relative effectiveness meeting system manager's or clinician's or patient's goals of different preventive and real-virtual interventions or care or supports, and so forth. As a result of this real-time information, analysis, and interpretation, health care managers assess goals for quality and costs and plan interventions to better achieve the goals of the health care system.

TABLE I
Implementation Levels 1-4
Features & Benefits

| | | Clinical and | Constraints to | | |
|---|---|---|---|---|---|
| | | Administrative | Implementation of | | |
| | Health | Information and | Additional Resources | Health System Features | Heath Care Benefits |
| Level | Authority Aims | Management Methods | at this Level | with Implementation | with Implementation |
| 0* | Quality Health | Current Health System | None Existing | Regulatory Drug | Scientific Medicine in |
| | Care | Resources | System | Approval for Efficacy | treatment selection but |
| | | | | and Safety | continued dependence |
| | | | | Evidence-based | on unsystematic clinical |
| | | | | Medicine (EBM) | judgments in patient |
| | | | | Grounding to | care |
| | | | | Treatment Choice | |
| | | | | Decisions | |
| | Cost Controls | | | Drug cost controls by | Costs vulnerable to |
| | | | | formulary restrictions, | introduction of new |
| | | | | negotiated prices, and | drugs, escalation of use |
| | | | | so forth. | by pharmaceutical |
| | | | | | promotions to |
| | | | | | practitioners and direct |
| | | | | | to consumer advertising |

*Level 0 is the current health care system without HP technology

| | | | | | |
|---|---|---|---|---|---|
| I* | Quality | Web-based Tools for | The methods of | Web based support | Quality patient care |
| | Improvement | determining treatment | invention provide- | allows physicians and | supported by web-based |
| | and | effectiveness in | Disease Management | patients to test | resources for Disease |
| | Cost savings | individuals | Supports for | effectiveness of drug | Management |
| | Through | Practitioners better | Practitioners | treatment with: | Interactive access for |
| | Precision and | manage disease | and | CIE*-EBM** criteria | Physicians supports:: |
| | Certainty Based | and | Informational and Self | CAE*** for non-CIE | EBM utilization; provides |
| | Disease | The Health Care System | Care Supports to the | patients. | precision and certainty in |
| | Management | better informs patients | Public | N-of-1 trials for | clinical examinations and |
| | Supports to | | | Dosing adjustments; | tests; integrates resources |
| | Practitioners and | | | Treatment comparisons | in convenient format |
| | the Public. | | | for maximum | promoting use by |
| | | | | effectiveness; Treatment | physicians; promotes more |
| | | | | comparisons for lowest | effective feedback controls |
| | | | | cost for equal | over system quality and |
| | | | | effectiveness | costs. |
| | | | | Web-based evidence of | Consumers fully informed |
| | | | | eligibility for | about personal care based |
| | | | | reimbursement of | on readily understood, |
| | | | | treatment costs | interpreted information on |
| | | | | | true clinical state. |

*CIE is a clinically important effect
**EBM is evidence-based medicine
***CAE is an effect clinically adequate to the patient's needs even though it is not CIE

| | | | | | |
|---|---|---|---|---|---|
| II | Web linked | Electronic, Real-Time, | Clinical management | Above plus: | A Self-learning System |
| | Information and | Self modulating, | Systems in place. | Aggregated data analysis | updates Health and |
| | Management | Information and | Information and | develops- | Disease Management |
| | System | Management System | Management System | Clinical Decision Rules | Models with Analysis and |
| | for | available to | aggregates data From | for Health and Disease | Interpretations of Clinical |
| | Effective Real- | Health Authorities for use | Health (Level IV) and | Management | Outcomes from the |
| | Time, Data- | in selected diseases and | Disease (Level II) | Clinical Decision Rules | system. Real-time, |
| | Based System | treatments | Management modules | Structure Drug Formulary | reliable feedback stabilizes |
| | Quality and Cost | | and analyzes data for | Approvals | system and improves |
| | Controls | | indicators of | Encourages | effectiveness of |
| | | | Quality and | Pharmaceutical | management interventions |
| | | | Cost savings | Companies to Provide | Improved hypothesis |
| | | | Analysis interprets data | Expanded Clinical Trial | generation for research |
| | | | for interventions for | data to speed | investigations |
| | | | Quality and | reimbursement for their | Allows more effective |
| | | | Cost Savings | drugs | implementation and |
| | | | | Expanded Pharmaceutical | economical use of |
| | | | | Clinical Trial data | Preventive Medicine and |
| | | | | supports Provincial or | Virtual Medicine. |
| | | | | Federal formulary | Provides Continuing |
| | | | | approvals with guides to | Education - Problem- |
| | | | | rational and economical | based Learning - to |
| | | | | uses of new drugs. | practitioners: in office; |
| | | | | | during patient care. |
| | | | | | All the above provide |
| | | | | | additional Quality |
| | | | | | improvements & Cost |
| | | | | | Savings |
| III | Balanced | Health Management | Disease Management | Citizen access to Self- | Improved quality. |
| | primary, | Tools with Level III | Systems and | Care Supports with | Improved cost controls. |
| | secondary, and | Monitoring of Self-care, | Information and | Monitors for | Quality Public Health |
| | tertiary | Direct care, and Virtual | Management Systems | effectiveness of choices | balance. |
| | preventive | Supports | in place | Citizen, practitioner, | Quality Virtual Care |
| | medicine. | for | | patient access to web | delivered |
| | | Effectiveness and | | based resources: | |
| | | Relative Costs | | Evidence-based | |
| | | | | summaries of medical | |
| | | | | knowledge | |
| | | | | Analysis and | |
| | | | | Interpretation Solutions | |
| | | | | Simulation for comparing | |
| | | | | possible decisions for | |
| | | | | probable outcomes | |
| IV | Quality | Design and Analysis of | Depends on | Current Regulatory | Pharmaceutical |
| | Improvement & | Clinical Trials to | Pharmaceutical | Approval for Efficacy | Companies provide |
| | Cost savings in | statistically and | Companies Utilizing | and Safety | Statistically Sound |
| | Treatment | scientifically inform | more Clinically | In addition: | Scientific Evidence to |
| | Management | clinicians' judgments | Informative Research | More Clinically | Support Guidelines for |
| | System: | about the effectiveness | Designs | Informative Clinical | Judging Effectiveness of |
| | Pharmaceutical | of drugs in individuals | and/or | Trial methods provide | New Drugs in Individual |
| | Manufacturers | | Regulatory revision | Clinical Decision Rules | Patients because |
| | in Clinical | | to implement | to guide patient | insurers, governments or |
| | Trials for | | Standards for Clinical | management for | other providers require |
| | proposed new | | Trials to demonstrate | specific diseases | this analysis before |
| | drug | | effectiveness in | | approving payments for |
| | demonstrate | | individual patients | | use of the drug in |
| | management to | | | | patients. |
| | identify | | | | |
| | individuals who | | | | |
| | benefit and | | | | |
| | those who do | | | | |
| | not form the | | | | |
| | proposed new | | | | |
| | therapy. | | | | |

What is claimed is:

1. A method of establishing health and disease assessments and using such assessments as grounds for applying health and disease management, comprising:
specifying predetermined health or medical aims for a health or disease management program;
selecting a method of assessing the specified aims;
determining a user specific error of measurement;
setting an assessment plan to provide reliability for descriptive summary statistics for an individual's course over time;
selecting criteria for achievement of health aims or for clinically significant health effects; and
identifying criteria of statistical significance.
